(19) United States
(12) Patent Application Publication
(10) Pub. No.: US 2024/0009392 A1
(43) Pub. Date: Jan. 11, 2024

(54) INFORMING A USER OF ANTICIPATED INSULIN DELIVERY ACTIONS AND PROVIDING RECOMMENDED USER ACTIONS

(71) Applicant: INSULET CORPORATION, Acton, MA (US)

(72) Inventors: Joon Bok LEE, Acton, MA (US); Joshua WILLIAMS, Marlborough, MA (US); Jason O'CONNOR, Acton, MA (US); Yibin ZHENG, Hartland, WI (US); Ashutosh ZADE, San Diego, CA (US)

(21) Appl. No.: 18/350,223

(22) Filed: Jul. 11, 2023

Related U.S. Application Data
(60) Provisional application No. 63/359,924, filed on Jul. 11, 2022.

(51) Int. Cl. A61M 5/172; A61M 5/142

(52) U.S. Cl. CPC A61M 5/1723; A61M 5/14244; A61M 2202/0486

(57) ABSTRACT

Exemplary embodiments may determine anticipated basal insulin delivery action to a user from an insulin delivery device over a future time window. Indications of the anticipated basal insulin delivery action over the future time window may be output to the user. The exemplary embodiments may determine the anticipated basal insulin delivery action over the future time window based on a rate of change (ROC) of glucose level of the user by the insulin delivery device, a most recent (“current”) glucose level for the user and insulin on board (IOB) for the user. The exemplary embodiments may also determine whether the user is likely to experience a undesired high glucose level (e.g., hyperglycemia) and/or an undesired low glucose level (e.g., hypoglycemia) during the future time window. The exemplary embodiments may output recommendations based on the projected glucose levels of the user over the future time window.

### RELATED APPLICATIONS

This application claims the benefit of U.S. Provisional Patent Application No. 63/359,924, filed Jul. 11, 2022, the entire contents of which are incorporated herein by reference in their entirety.

### BACKGROUND

Users of automatic insulin delivery (AID) devices are often not fully aware of what amounts of insulin are projected to be delivered to the users by the AID devices in an upcoming time window. As a result, the users may make decisions that produce less than optimal results. For example, a user may believe that an insulin bolus is needed to lower the glucose level of the user without realizing that the projected basal insulin deliveries are sufficient to lower the glucose level of the user to a safe and acceptable level in the time window. The unnecessary insulin bolus may place the user at risk of hypoglycemia. In another example, a user may believe that the projected insulin deliveries in the upcoming time window will not pose an issue to the user. However, if less insulin is delivered in the upcoming time window than the user anticipates, the glucose level of the user may rise to exceed the hyperglycemic threshold or may be at an undesired elevated level below the hyperglycemic threshold.

### SUMMARY

In accordance with an inventive facet, an electronic device includes a display for displaying content and a processor. The processor is configured to determine projected basal insulin delivery amounts by an automated insulin delivery (AID) device to the user over an interval based on a current rate of change of basal insulin delivery by the AID device to a user, a current amount of insulin on board for the user and a most recent glucose level reading for the user. The processor is also configured to output an indication of the projected basal insulin delivery amounts by the AID device to the user over the interval on the display.

The indication may include a description of the projected basal insulin delivery amounts by the AID device to the user over the interval. The description may indicate at least one of the following: that basal insulin delivery to the user will be suspended during at least a portion of the interval, that basal insulin delivery amounts to the user during the interval will be substantially equal to a current basal insulin delivery amount delivered to the user, the basal insulin delivery to the user will be increased during the interval to increased amounts relative to the current basal insulin delivery amount delivered to the user, or the basal insulin delivery amounts delivered to the user will be significantly increased to significantly increased amounts during the interval relative to current basal insulin delivery amount delivered to the user. The indication may include graphical content.

In determining the projected insulin delivery by the AID device to the user over an interval, the processor may be configured to compare a current rate of change (RoC) of the glucose level of the user to one or more thresholds, compare the current amount of insulin on board for the user and the most recent glucose level reading for the user to thresholds, and based on the comparing of the current RoC, the comparing of the current amount insulin on board and the comparing of the most recent glucose level reading for the user, determine the projected insulin delivery amounts by the AID device to the user. The processor may be further configured to determine anticipated glucose levels for the user over the interval in view of the projected basal insulin delivery amounts. The processor may additionally be configured to determine whether the anticipated glucose levels for the user over the interval will exceed a desired range of glucose levels. The processor may additionally be configured to notify the user that the anticipated glucose levels for the user over the interval will exceed the desired range of glucose levels. The processor may be additionally configured to determine whether the anticipated glucose levels of the user over the interval will fall below the desired range of glucose levels. The processor may be additionally configured to notify the user that the anticipated glucose levels for the user over the interval will fall below the desired range of glucose levels. The interval may extend from a current time to a time in the future. The time in the future may be a time in a range of 15 minutes after the current time to two hours after the current time.

In accordance with another inventive facet, an electronic device includes a display for displaying content and a processor configured to determine projected insulin delivery amounts by an automatic insulin delivery (AID) device to a user over a future interval. The processor is further configured to determine if anticipated glucose levels of the user for the future interval stay within a desired range, and if the anticipated glucose levels of the user for the interval are determined to not stay within the desired range, to output a recommendation on the display for action by the user to adjust their glucose level.

The recommendation may be for the user to receive a correction insulin bolus or for the user to consume carbohydrates. The electronic device may be at least one of a management device for the AID device, the AID device, a smartphone, a wearable device or a handheld device.

In accordance with an additional inventive facet, a method is performed by a processor of an electronic device that includes a display. The method entails determining projected basal insulin delivery amounts by an automatic insulin delivery (AID) device to a user over a future time window and determining anticipated glucose levels of the user over the future time window in view of the determined projected basal insulin delivery amounts. The method further includes displaying a user interface on the display. The user interface includes graphical content regarding the anticipated glucose levels and a visual cue of the projected basal insulin delivery amounts to inform the user of the projected basal insulin delivery amounts.

The determining of the projected basal insulin delivery amounts may be based on a current glucose level of the user, a current rate of change of the basal insulin delivery amounts and total daily insulin for the user. The method additionally may include determining that at least some of the anticipated glucose levels over the future time window will exceed a threshold and in response, outputting a recommendation on the display of action that the user may take to reduce the anticipated glucose levels over the future time window. The method further may include determining that at least some of the anticipated glucose levels over the future time window will fall below a threshold and in response, outputting a recommendation on the display of action that the user may take to increase the anticipated glucose levels over the future time window.

### BRIEF DESCRIPTION OF THE DRAWINGS

FIG. 1 depicts an illustrative medicament delivery system suitable for exemplary embodiments.

FIG. 2A depicts a flowchart of illustrative steps that may be performed in exemplary embodiments to display anticipated insulin delivery action for a user over a time window.

FIG. 2B depicts an example of a user interface that may be displayed in exemplary embodiments to communicate the anticipated insulin delivery action for a user over a time window.

FIG. 2C depicts an example of a user interface that may be displayed in exemplary embodiments to communicate the anticipated insulin delivery action for a user over a time window on a home screen.

FIG. 3 depicts a flowchart of illustrative steps that may be performed in exemplary embodiments to determine projected insulin delivery amounts to the user over the time window.

FIG. 4 depicts a flowchart of illustrative steps that may be performed in exemplary embodiments to apply the highest RoC rules.

FIG. 5 depicts a flowchart of illustrative steps that may be performed in exemplary embodiments to apply the high RoC rules.

FIG. 6 depicts a flowchart of illustrative steps that may be performed in exemplary embodiments to apply the low negative RoC rules.

FIG. 7 depicts a flowchart of illustrative steps that may be performed in exemplary embodiments to apply the lowest negative RoC rules.

FIG. 8 depicts a flowchart of illustrative steps that may be performed in exemplary embodiments to additionally facilitate the use of recommendations related to glucose levels of the user.

FIG. 9 depicts a flowchart of illustrative steps that may be performed in exemplary embodiments to provide a recommendation to the user to take action.

FIG. 10A depicts a flowchart of illustrative steps that may be performed in exemplary embodiments to determine if the user will experience high glucose conditions during the time window.

FIG. 10B depicts an illustrative recommendation for an exemplary embodiment recommending that the user to deliver an insulin bolus.

FIG. 11A depicts a flowchart of illustrative steps that may be performed in exemplary embodiments to determine if the user will experience low glucose conditions during the time window.

FIG. 11B depicts an illustrative recommendation for an exemplary embodiment recommending that the user to ingest rescue carbohydrates.

FIG. 12 depicts a flowchart of illustrative steps that may be performed in exemplary embodiments to identify and respond to an indication of glucose resistance in the user found in glucose levels of the user.

FIG. 13 depicts an illustrative user interface that may be displayed in exemplary embodiments to prompt a use to exercise responsive to an elevated glucose indicative of insulin resistance.

FIG. 14 depicts a flowchart of illustrative steps that may be performed in exemplary embodiments to identify and respond to an indication of glucose resistance in the user found in RoC of glucose levels of the user.

FIG. 15 depicts an illustrative user interface that may be displayed in exemplary embodiments to prompt a use to exercise responsive to a RoC of glucose level indicative of insulin resistance.

### DETAILED DESCRIPTION

Exemplary embodiments described herein may determine anticipated basal insulin delivery action to a user from an insulin delivery device over a future time window. Indications of the anticipated basal insulin delivery action over the future time window may be output to the user. With this knowledge of the anticipated basal insulin delivery action, the user may take action to avoid undesirable glucose levels and more generally take into account the anticipated basal insulin delivery action over the future time window. This knowledge may eliminate uncertainty that the user may have as to future basal insulin deliveries over the future time window and may serve to provide a degree of comfort to the user.

The exemplary embodiments may determine the anticipated basal insulin delivery action over the future time window based on a rate of change (RoC) in a glucose level of the user by the insulin delivery device, a most recent (“current”) glucose level for the user and insulin on board (JOB) for the user. The exemplary embodiments may be able to project the response of the control system of the insulin delivery device based on the RoC, current glucose level and IOB to determine the anticipated basal insulin delivery action over the future time window.

The exemplary embodiments may also determine whether the user is likely to experience a undesired high glucose level (e.g., hyperglycemia) and/or an undesired low glucose level (e.g., hypoglycemia) during the future time window. This determination may be made from the RoC, the current glucose level of the user and the current IOB of the user. The exemplary embodiments may output recommendations to the user based on the projected glucose levels of the user over the future time window. For example, the user may be issued a recommendation to take action to increase their glucose level or may be issued a recommendation to take action to decrease their glucose level.

Parties that receive exogenous insulin, such as parties that use insulin delivery devices, may be prone to developing insulin resistance. As a result, such parties may require increasing amounts of insulin, may exhibit higher levels of glucose and may gain weight. The exemplary embodiments may determine whether a user of an insulin delivery device is exhibiting insulin resistance and may generate a notification to the user to prompt the user to take action to increase the user's insulin sensitivity. The exemplary embodiments may monitor the glucose level of the user after receiving insulin, such as a bolus. The exemplary embodiments may predict what glucose level of the user should be a period of time after delivery of the insulin and may compare the predicted glucose level with an actual glucose level. If the actual glucose is more than a threshold amount greater than the predicted glucose level, a notification may be sent to the user. The notification may, for instance, prompt the user to exercise, which is known to increase insulin sensitivity. In some alternative embodiments, the RoC of the glucose level may be monitored rather than the glucose level. If the RoC is not great enough, a notification may be sent to the user.

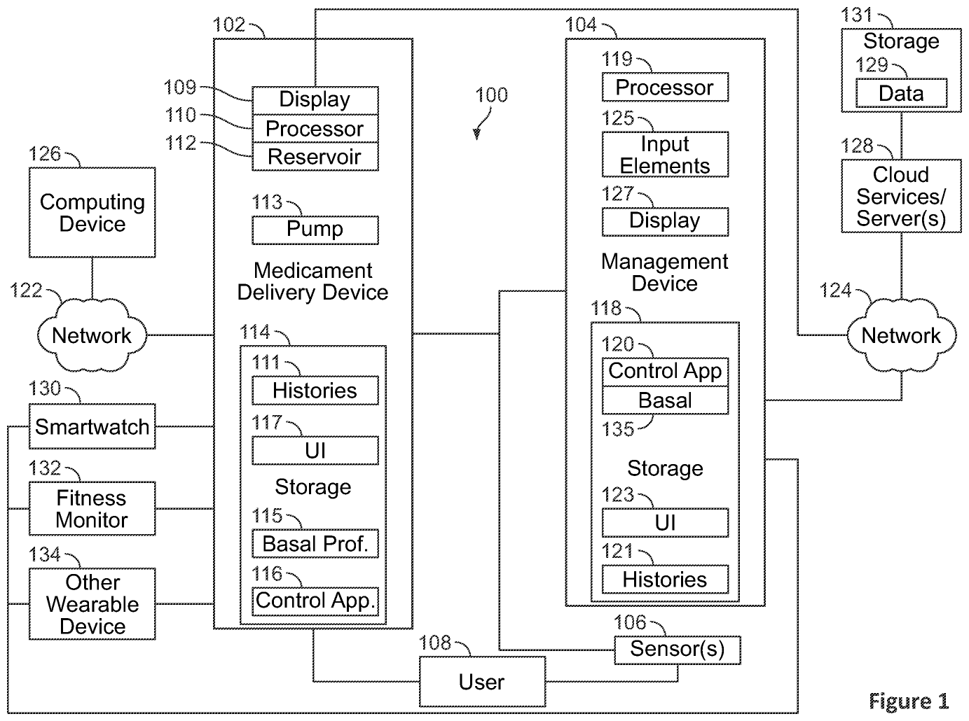

FIG. 1 depicts an illustrative medicament delivery system 100 that is suitable for delivering a medicament to a user 108 in accordance with the exemplary embodiments. The medicament delivery system 100 includes a medicament delivery device 102, such as an insulin delivery device like an automatic insulin delivery (AID) device. The medicament delivery device 102 may be a wearable device that is worn on the body of the user 108 or carried by the user. The medicament delivery device 102 may be directly coupled to a user (e.g., directly attached to a body part and/or skin of the user 108 via an adhesive or the like) or carried by the user (e.g., on a belt or in a pocket) with the medicament delivery device 102 being connected to an infusion site where the medicament is injected using a needle and/or cannula. In a preferred embodiment, a surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries, or the like. The control application 116 may be responsible for implementing the control loop that provides feedback and adjustments to medicament dosages that are delivered to a user. The processor 110 also may execute computer programming instructions stored in the storage 114 for a user interface 117 that may include one or more display screens shown on display 109. The display 109 may display information to the user 108 and, in some instances, may receive input from the user 108, such as when the display 109 is a touchscreen.

The control application 116 may control delivery of a medicament to the user 108 per a control approach like that described herein. The storage 114 may hold histories 111 for a user, such as a history of basal deliveries, a history of bolus deliveries, and/or other histories, such as a meal event history, exercise event history, glucose level history and/or the like. These histories may be processed as will be described below to adjust basal medicament dosages to help reduce or eliminate persistent positive low level medicament excursions. The storage 114 also may include one or more basal profiles 115 that are used when the medicament delivery device is operating in open loop mode. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include one or more housings for housing its various components including a pump 113, a power source (not shown), and a reservoir 112 for storing a medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112) and may include a conduit to a separate infusion site.

There may be one or more communications links with one or more devices physically separated from the medicament delivery device 102 including, for example, a management device 104 of the user and/or a caregiver of the user, a sensor 106, a smartwatch 130, a fitness monitor 132 and/or another variety of wearable device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN) or a combination therein. A computing device 126 may be interfaced with the network, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions and/or physical attributes of the user 108. The sensor(s) 106 may be physically separate from the medicament delivery device 102 or may be an integrated component thereof.

The medicament delivery system 100 may or may not also include a management device 104. In some embodiments, no management device is not needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensors 106. The management device 104 may be any portable electronic device including, for example, a dedicated device, a smartphone, a smartwatch or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store control application 120 for execution by the processor 119. The control application 120 may be responsible for controlling the medicament delivery device 102, such as by controlling the AID delivery of insulin to the user 108. The storage 118 may store the one or more control applications 120, histories 121 like those described above for the medicament delivery device 102, one or more basal profiles 135 and other data and/or programs.

A display 127, such as a touchscreen, may be provided for displaying information. The display 127 may display user interface (UI) 123. The display 127 also may be used to receive input, such as when it is a touchscreen. The management device 104 may further include input elements 125, such as a keyboard, button, knobs, or the like, for receiving input form the user 108.

The management device 104 may interface with a network 124, such as a LAN or WAN or combination of such networks via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128. The cloud services/server(s) 128 may provide output from the model 115 as needed to the management device 104 and/or medicament delivery device 102 during operation.

Other devices, like smartwatch 130, fitness monitor 132 and wearable device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user, such as to request a change or pause in dosage or to request, initiate, or confirm delivery of a bolus of a medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data.

The functionality described below for the exemplary embodiments may be under the control of or performed by the control application 116 of the medicament delivery device 102 or the control application 120 of the management device 104. In some embodiments, the functionality may be under the control of or performed by the cloud services or servers 128, the computing device 126 or by the other enumerated devices, including smartwatch 130, fitness monitor 132 or another wearable device 134.

The medicament delivery device 102 may operate in an open loop mode and in a closed loop mode. In the open loop mode, the user 108 manually inputs the amount of medicament to be delivered (such as per hour) for segments of the day. The inputs may be stored in a basal profile 115, 135 for the user 108. In other embodiments, a basal profile may not be used. The control application 116, 120 uses the input information from the basal profile 115, 135 to control basal medicament deliveries in open loop mode. In contrast, in the closed loop mode, the control application 116, 120 determines the medicant delivery amount for the user 108 on an ongoing basis based on a feedback loop. For an insulin delivery device, the aim of the closed loop mode is to have the user's glucose level at a target glucose level. The basal dosages may be delivered at fixed regular intervals, designated as cycles, such as every five minutes.

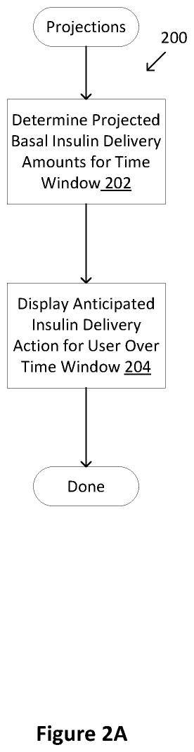

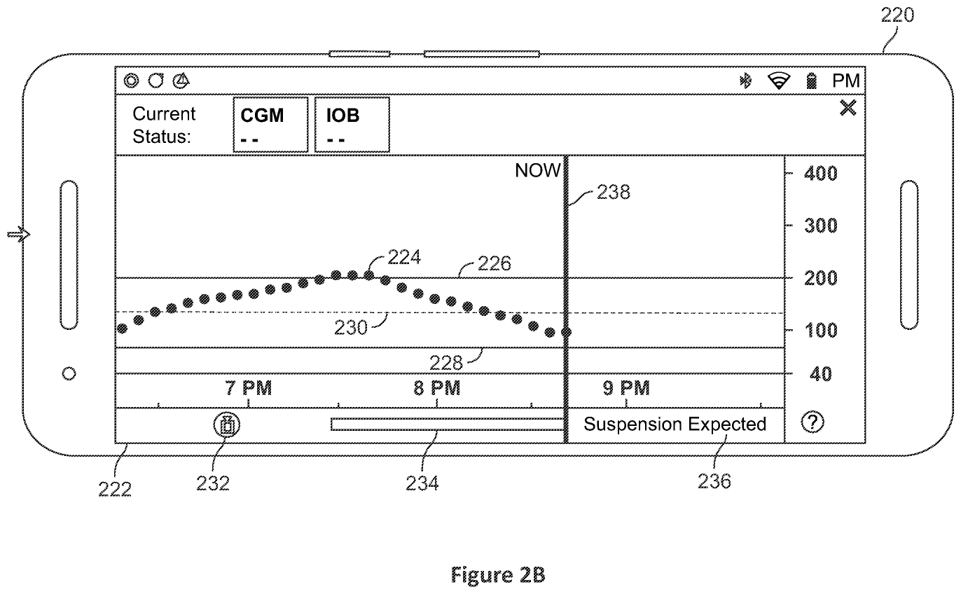

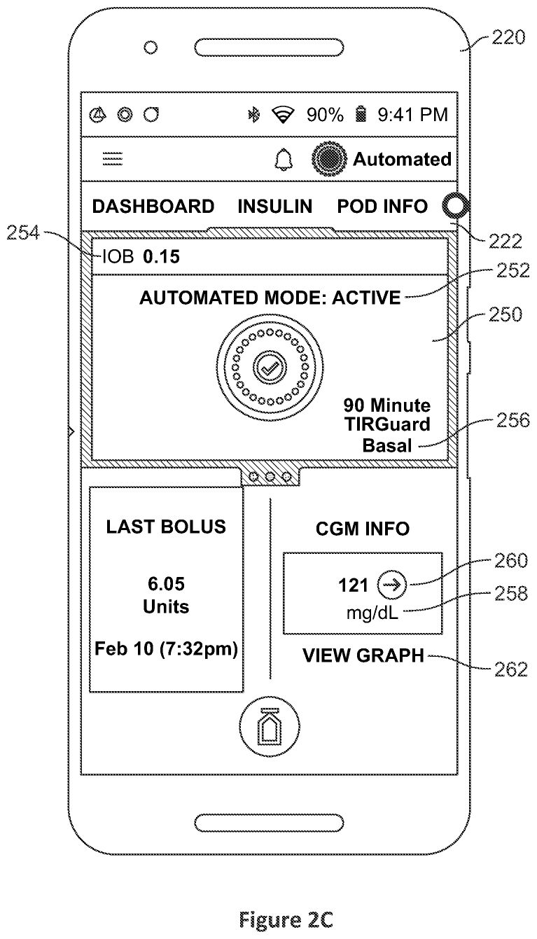

FIG. 2A depicts a flowchart 200 of illustrative steps that may be performed in exemplary embodiments regarding projected basal insulin delivery amount to the user 108 from the medicament delivery device 102 over a time window operating in a closed loop mode. The time window may begin at a current time and extend to an end time, such as, for instance, 60 minutes or 90 minutes. The length of the time window may be variable but likely is more useful when the time window spans at least 15 minutes. At 202, projected basal insulin delivery amounts to the user 108 over the time window are determined, as will be described below. In view of the projected insulin delivery amounts for the time window, an anticipated insulin delivery action for the user over the time window may be displayed, such as on display 127 of the management device, display 109 of the medicament delivery device and/or on a display of the smartwatch 130, fitness monitor 132 or other wearable device 134. The display of the anticipated basal insulin delivery action for the user over the time window informs the user 108 of the anticipated basal insulin delivery action during at least a portion of the time window. For example, the anticipated basal insulin delivery action may be that the basal deliveries of insulin from the medicament delivery device 102 is anticipated to stay substantially at their current amounts and delivery rate. Alternatively, the anticipated basal insulin delivery action may be that insulin delivery from the medicament delivery device may be suspended for at least a portion of the time window. Still further, the anticipated basal insulin delivery action over the time window may be that the insulin delivery to the user 108 from the medicament delivery device 102 may be one of significantly increased, slightly increased, slightly decreased or significantly decreased. The granularity of the anticipated basal insulin delivery actions for the insulin delivery actions may be varied and customized.

The display of the anticipated basal insulin delivery action helps the user 108 to be informed of what will happen over the time window. The user 108 can then react to the anticipated insulin delivery action. For example, the user may avoid delivering an insulin bolus if the user 108 realizes that basal insulin delivery will increase over the time window so that no bolus is needed. As another example, the user 108 may avoid a snack where the user sees that the insulin delivery action is to decrease basal insulin delivery over the time window. More generally, the user may take other actions, such as ones that avoid risks, responsive to the knowledge of the anticipated insulin delivery action. In addition, the user may be comforted by knowing what the anticipated insulin delivery action is over the time window. In addition, the user may gain insights from the display of the anticipated basal insulin delivery action.

It should be appreciated that there may be multiple anticipated basal insulin delivery actions for the time window not just one. The multiple anticipated basal insulin delivery actions may be displayed on a display and an indication of when the actions occur in the time window may be displayed on the display as well.

FIG. 2B depicts an example of a user interface on the display 127 of the management device 102 that contains information regarding the anticipated basal insulin delivery action for a time window. In this instance, the management device 102 is a smartphone 220 that has a display 222. The display 222 shows a plot 224 of historical glucose values for the user 108. The graphical display also contains a high threshold 226 (of roughly 200 mg/dL) for glucose levels and a low threshold 228 (of roughly 80 mg/dL). A target level 230 (of roughly 120 md/dL) is also displayed. A bar 238 represents the current time of 8:41 PM. A vial icon 232 indicates that an insulin bolus was delivered to the user 108 shortly before 7:00 PM. A bar 234 indicates that basal insulin delivery has been suspended since shortly before 7:30 PM due to the decrease in glucose levels resulting from the insulin bolus. An additional bar 236 specifies that anticipated insulin delivery action for the time window of 90 minutes is continued basal insulin delivery suspension due to the glucose trend and low glucose trend. This user interface may be generated by and the logic required for the user interface may be performed by, for instance, the control application 120 or the control application 116.

FIG. 2C depicts another example of a user interface that may be displayed to convey the anticipated basal insulin delivery action over the time window. The user interface of FIG. 2C is displayed on display 222 of the management device 220. An upper frame 250 show that the automated mode is active 252 and that the anticipated basal insulin delivery action for the time window is that insulin will be delivered at substantially the standard basal delivery rate 256. The upper frame 250 also shows the IOB 254 for the user 108. The lower right panel show the most recent glucose level reading 258 for the user and a glucose trend icon 260 indicating that glucose level trend is flat so that it is not increasing or decreasing to any notable degree. Element 261 may be selected to display the user interface of FIG. 2B.

It should be appreciated that the user interfaces of FIGS. 2B and 2C are intended to be merely illustrative and not limiting. Other user interfaces may be used to display the anticipated insulin delivery action for the time window.

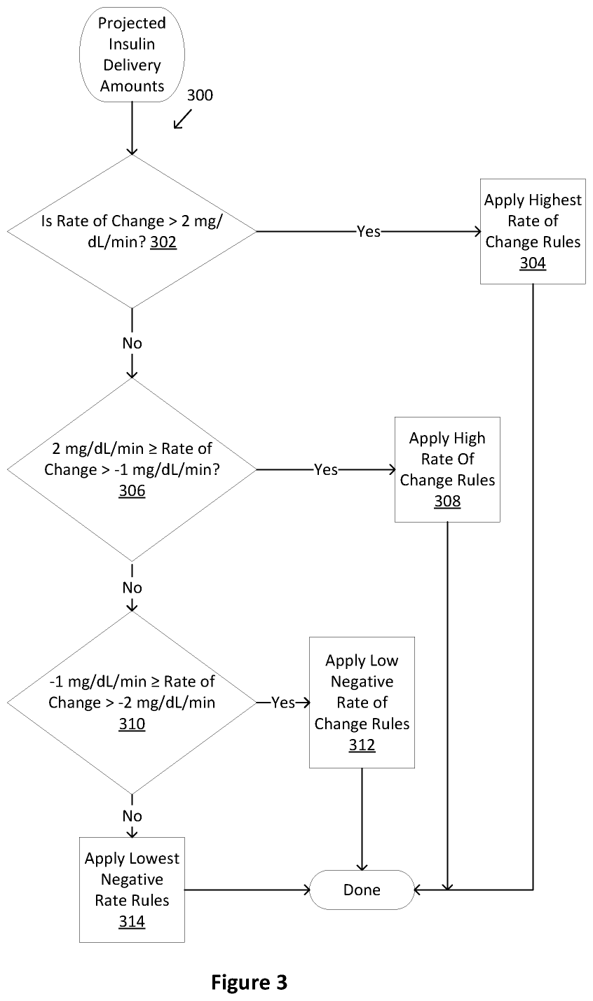

FIG. 3 depicts a flowchart 300 of illustrative steps that may be performed to determine the projected insulin delivery amounts to the user 108 over the time window (see 202 in FIG. 2A). The exemplary embodiments may look at the RoC of the glucose level of the user, the latest glucose level for the user 108 and the magnitude of the IOB for the user to determine the projected insulin delivery amounts to the user 108 over the time window. A cycle may represent a period of time, such as 5 minutes, where the basal insulin is determined and delivered to the user 108 (e.g., every 5 minutes). The RoC of the glucose level of the user may be calculated as the difference between the glucose level of the user for the current cycle and the glucose level for the user for three cycles before the current cycle. Alternatively, the RoC may be calculated between successive cycles in the time window and the average RoC may be used as the RoC.

The determination of the projected basal insulin delivery amounts varies based on the RoC of the glucose level of the user. Thus, at 302, a check is made whether the RoC of the glucose level of the user is greater than 2 mg/dL/min. The RoC of 2 mg/dL/min constitutes a highest RoC threshold for the control system and as such, highest RoC rules are applied at 304 if the RoC is above that threshold. If not, at 306, a check is made whether the ROC is between 2 mg/dL/min and −1 mg/dL/min. If so, high RoC rules are applied at 308. If not, a check is performed to see if the RoC is less than −1 mg/dL/min and −2 md/dL/min at 310. If so, the low negative RoC rules are applied at 312. If not, it means that the RoC is less than −2 mg/dL/min, and the lowest negative rate rules are applied at 314.

These projected insulin delivery amounts can vary significantly based on the design of the AID systems. For instance, the various thresholds of RoC to apply different rate of change rules may instead be different values, such as 2.5, 3, 3.5, or 4 mg/dL/min or greater in the positive direction, and −2.5, 3, 3.5, or 4 mg/dL/min or lower in the negative direction. Alternately, these ROC thresholds may also be applied to a sign-based projection where the only delineation is between a positive RoC value above a threshold, or a negative RoC value below a threshold.

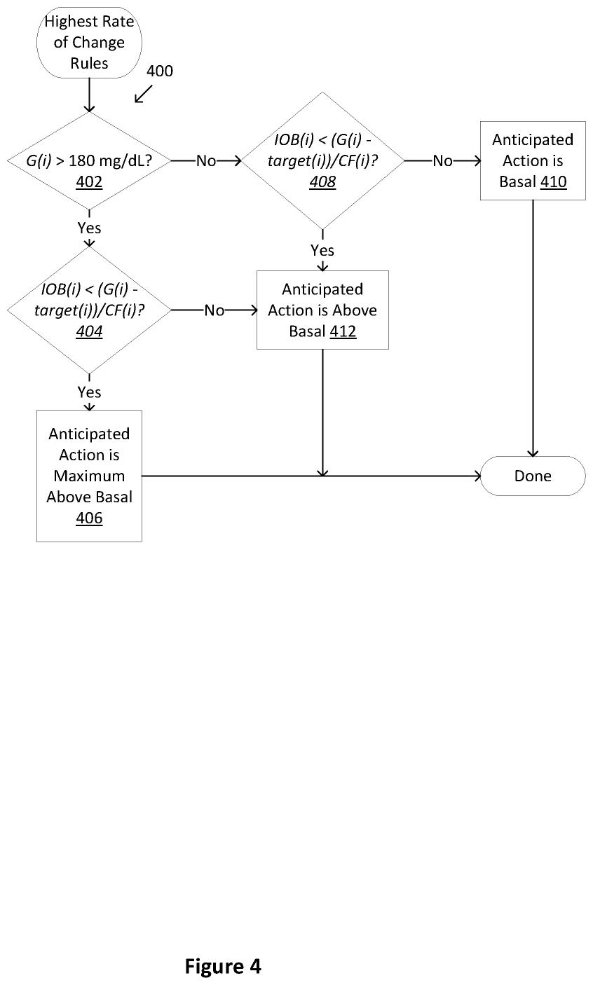

FIG. 4 depicts a flowchart 400 of illustrative steps that may be performed in exemplary embodiments to apply the highest RoC rules. At 402, the glucose level of the user G(i) 108 for the current cycle i is compared to a high threshold of 180 mg/dL. If the glucose level of the user G(i) is above 180 mg/dL, at 404, a check if made whether the IOB for the user 108 is less than (G(i)−target(i))/CF(i), where target(i) is the glucose level target for the user 108 at cycle i and CF(i) is the correction factor for the user at cycle i. The correction factor is how much 1 unit of insulin will lower a user's blood glucose over 2 to 4 hours when the user is in a fasting or pre-meal state. Thus, 404 checks whether the IOB for the user 108 is less than how much the current glucose level of the user 108 is above target divided by the correction factor. This comparison checks whether the IOB is sufficient to compensate for the current excess glucose level above target(i). If not, at 404, the insulin rate is too high, and there is not enough IOB to compensate. Hence, at 406, the anticipated basal insulin delivery action is projected to be delivery at a maximum above basal rate for the time window. If so, at 412, the basal insulin delivery rate is projected to be increased but not at the highest rate. Instead, the anticipated basal insulin delivery action for the time window is projected to be delivery at above the current basal rate. Similarly, at 408, if the glucose level of the user 108 G(i) is not above 180 mg/dL, and the IOB cannot compensate for the excess glucose level of the user above target (i.e., IOB(i)<(G(i)−target(i))/CF(i)), at 412, the anticipated basal insulin delivery action is delivery at above the current basal rate. If IOB(i) is not <(G(i)−target(i))/CF(i)), the anticipated basal insulin delivery action is delivery at the basal delivery rate, at 410, because the glucose level of the user is not too high and the IOB can compensate for the excess glucose.

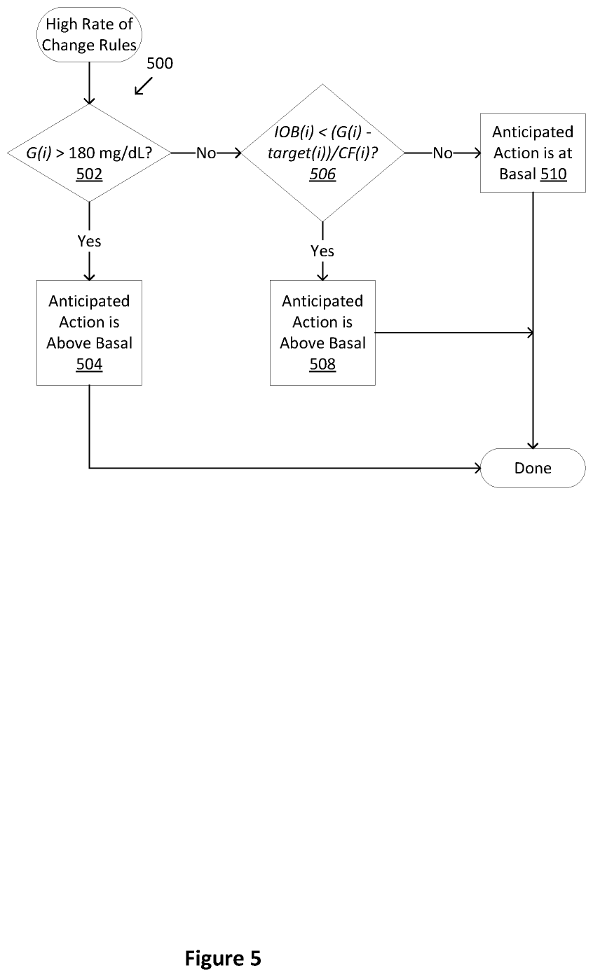

FIG. 5 depicts a flowchart 500 of illustrative steps that may be performed in exemplary embodiments to apply the high RoC rules. At 502, the current glucose level of the user 108 G(i) is compared to a high threshold of 180 mg/dL. If G(i) is above 180 mg/dL, at 504, the anticipated insulin delivery action is determined to be delivery at above basal to compensate for the high current glucose level G(i). If G(i) is not above 180 mg/dL, at 506, a check is made whether IOB(i)<(G(i)−target(i))/CF(i)). If so, at 508, the anticipated basal insulin delivery action is determined to be delivery at above basal because the IOB cannot compensate for the high glucose level that is not above 180 mg/dL but still is elevated. If not, at 510, the anticipated basal insulin delivery action is determined to be delivery at the basal rate because there is not a need for an above basal insulin delivery rate.

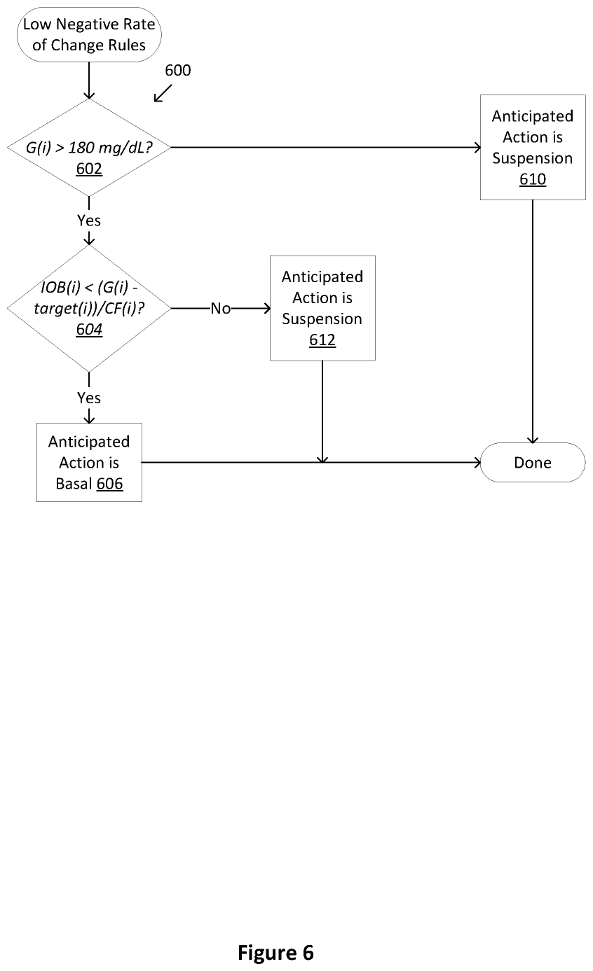

FIG. 6 depicts a flowchart 600 of illustrative steps that may be performed in exemplary embodiments to apply the low negative RoC rules. These rules are applied when the RoC of the glucose level of the user is negative but not greatly so. At 602, a check is made whether G(i) is greater than 180 mg/dL. If so, at 604, a check is made whether IOB(i)<(G(i)−target(i))/CF(i)). If so, at 606, the anticipated basal insulin delivery action is determined to be delivery at the current basal delivery rate. This is because the current glucose level for the user is not elevated and the IOB is not excessive given that the RoC is at a low negative rate. If at 602, it is determined that G(i) is not greater than 180 mg/dL, then at 608, the anticipated basal insulin delivery faction or the time window is suspension of delivery. If at 604, it is determined that whether IOB(i)≥(G(i)−target(i))/CF(i)), the anticipate basal insulin delivery action is suspension of delivery.

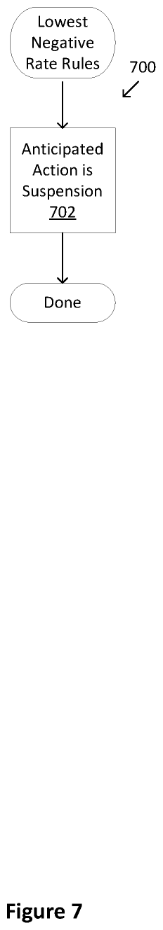

FIG. 7 depicts a flowchart 700 of illustrative steps that may be performed in exemplary embodiments to apply the lowest negative RoC rules. If the lowest negative RoC rules are applied, the anticipated basal insulin delivery action is suspension of basal insulin delivery at 702. This is because such a sharply negative RoC indicates that a suspension is imminent.

It should be appreciated that the high glucose level threshold that is checked in some of the above flowcharts may be chosen as a different value than 180 mg/dL. For instance, the high glucose level threshold may be 200 mg/dL or 175 mg/dL. Likewise the RoC thresholds in 302, 306 and 301 may differ from the RoCs shown.

Various values may be chosen for the above basal insulin delivery rate and the maximum above basal insulin delivery rate. Suitable values are two times the basal delivery rate of the user 108 as the above basal insulin delivery rate and four times the basal delivery rate of the user as the maximum above basal insulin delivery rate.

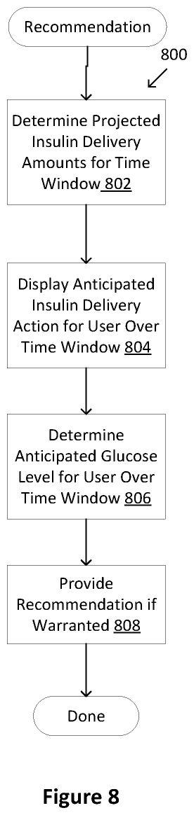

The exemplary embodiments may also provide recommendations for user action in view of the anticipated insulin delivery action for the user 108 over the time window. FIG. 8 depicts a flowchart 800 of illustrative steps that may be performed in exemplary embodiments for such exemplary embodiments. At 802, the projected insulin delivery amounts for the time window are determined as described above. At 804, the anticipated insulin delivery action for the user 108 over the time window is determined as described above. At 806, anticipated glucose levels for the user 108 over the time window in view of the projected insulin delivery amounts are determined. At 808, based on these anticipated glucose levels, one or more recommendations are provided to the user 108 if warranted. The recommendations are described in more detail below.

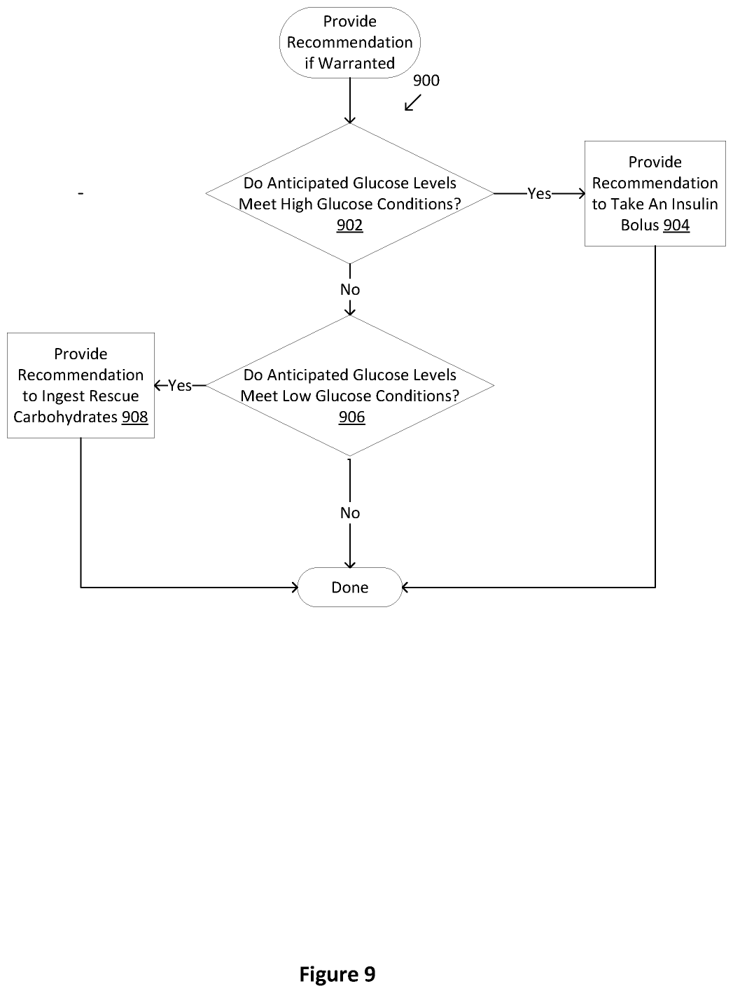

FIG. 9 depicts a flowchart 900 of illustrative steps that may be performed in exemplary embodiments in providing a recommendation if warranted (see 808). The anticipated glucose levels can be determined given the current glucose level of the user 108, the anticipated insulin delivery action for the user over the time window and the RoC. If the user meets high glucose conditions as checked at 902, a recommendation to take an insulin bolus may be output to the user 108, such as on a display, at 904.

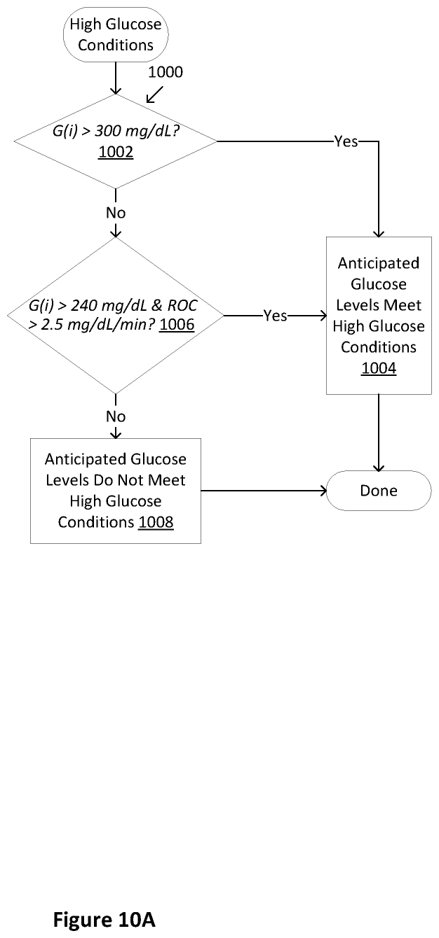

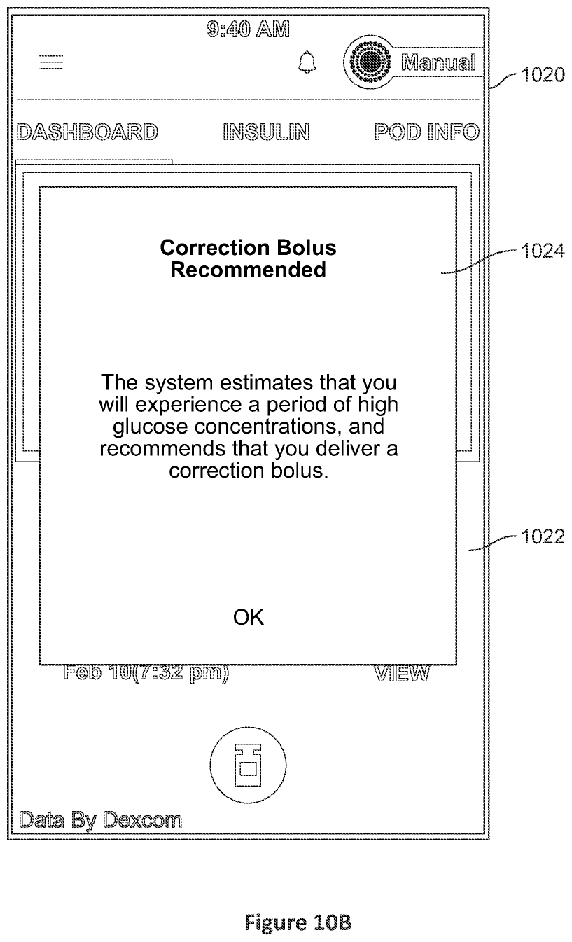

FIG. 10A depicts a flowchart 1000 of illustrative steps that may be performed to determine if the anticipated glucose levels for the user meet high glucose conditions (see 902). At 1002, it is determined whether G(i) is greater than 300 mg/dL (a highly hyperglycemic level). If G(i) is greater than 300 mg/dL, than it is determined that the user 108 is experiencing high glucose conditions at 1004, and a recommendation may be generated in response. If G(i) is less than 300 mg/dL, a determination is made whether G(i) is greater than 240 mg/dL (a lower hyperglycemic level) and whether the RoC of the glucose level of the user 108 is greater than 2.5 mg/dL/min at 1006. If so, at 1004, it is determined that the anticipated glucose levels for the user 108 meet high glucose conditions. If not, at 1008, it is determined that the anticipated glucose levels for the user 108 over the time window do not meet high glucose conditions and hence, a recommendation is not needed.

As in the previous embodiments, the threshold for high glucose concentrations for this anticipation, such as 300 or 250 mg/dL, are tunable parameters. These parameters can be modified to a wide range of values as desired per application.

FIG. 10B depicts an example of a recommendation that may be provided at 904. The recommendation 1024 is shown on the display 1022 of the management device 1020. The recommendation contains text encouraging the user 108 to deliver an insulin bolus to lower his/her glucose level. In some exemplary embodiments, the recommendation 1024 may also be accompanied by audio output or the recommendation can be a video. The recommendation could, instead or in addition to recommending the delivery of the insulin bolus, recommend that the user 108 exercise to reduce their glucose level.

With respect to FIG. 9, if at 902, it is determined that the anticipated glucose levels for the user 108 over the time window do not meet high glucose conditions, a check is made whether the anticipated glucose levels for the user 108 over the time window meet low glucose conditions at 906. If so, at 908, a recommendation may be output to the user to ingest rescue carbohydrates to raise the glucose level of the user 108. If not, no recommendation is output.

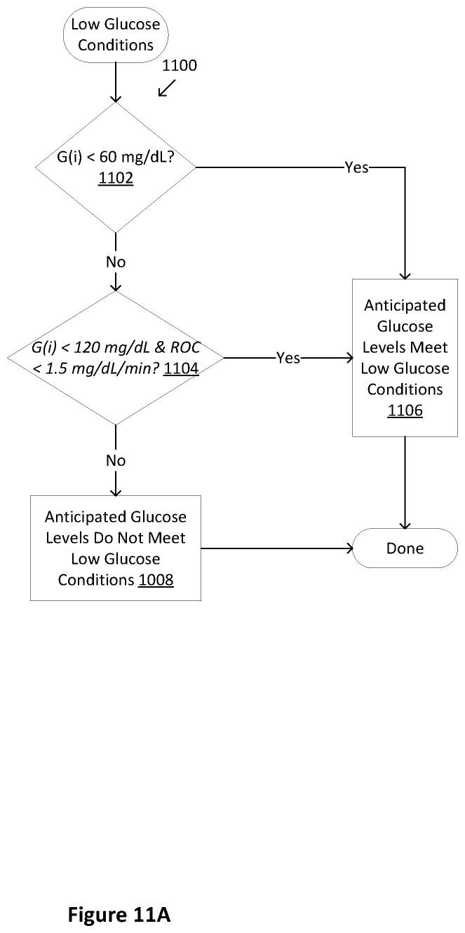

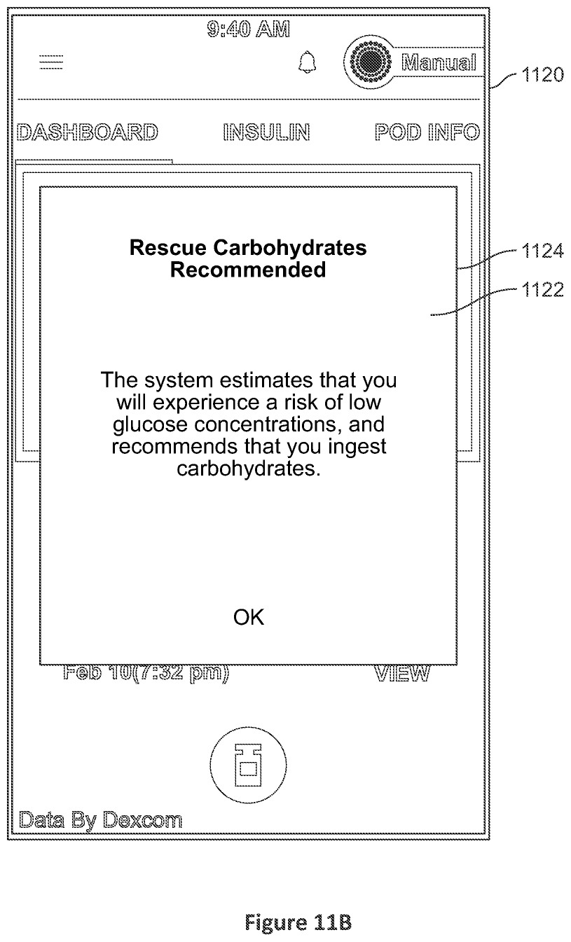

FIG. 11A depicts a flowchart 1100 of illustrative steps that may be performed in exemplary embodiments to determine if the anticipated glucose levels of the user 108 meet low glucose conditions (see 906). At 1102, a check is made to determine if G(i) is less than 60 mg/dL (a hypoglycemic level). If so, the user 108 is anticipated to be at hypoglycemic levels in the time window, and at 1106, it is determined that the anticipated glucose levels for the user meet low glucose conditions. If not, at 1104, a check is made whether G(i) is less than 120 mg/dL (a target glucose level) and the current RoC of the glucose level of the user is less than 1.5 mg/dL/min. If so, the anticipated glucose levels for the user over the time window are determined to meet low glucose conditions at 1106. If not, the anticipated glucose levels for the user over the time window are determined to not meet low glucose conditions at 1108.

FIG. 11B shows an example of a recommendation for a user to ingest rescue carbohydrates responsive to anticipated low glucose conditions during the time window. The recommendation 1122 is displayed on display 1124 of management device 1120. The recommendation 1122 contains text recommending ingestion of rescue carbohydrates. The recommendation may be realized as video and may be accompanied or replaced by an audio message. In some alternative embodiments, the user 108 may receive a recommendation to deliver a bolus of glucagon to raise their glucose level.

It should be appreciated that different threshold values may be used in 1002, 1006, 1102 and 1104.

As was mentioned above, the exemplary embodiments may identify patterns associated with insulin resistance and may send notifications to a user to attempt to counter insulin resistance. The patterns may be found, in glucose levels of the user, RoC in glucose levels of the user or even in acceleration or deceleration of the RoC in glucose levels of the user.

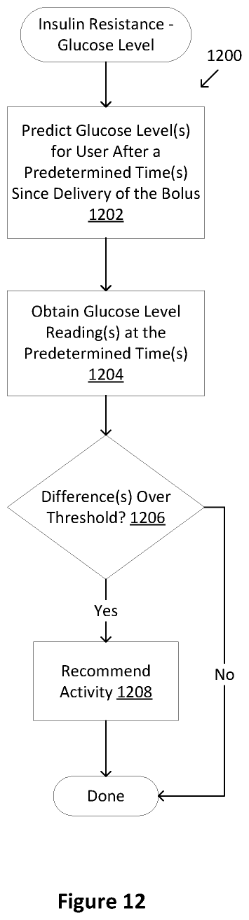

FIG. 12 depicts a flowchart 1200 of illustrative steps that may be performed in exemplary embodiments to identify and respond to an indication of glucose resistance in the user found in glucose levels of the user. Typically, insulin resistance may be evidenced by a glucose level of a user remaining elevated after a period of time after delivery of a correction insulin bolus when the glucose level should not be so elevated. The period of time may be the time for it takes the bolus to have full effect or a period of time where the anticipated effect of the insulin bolus is known. The period of time may vary depending on type of insulin. Typically rapid acting insulin is used for correction boluses. At 1202, one or more predicted glucose level(s) for the user at a predetermined time or times after the delivery of a correction insulin bolus is/are determined. When multiple predicted glucose levels are determined, the predicted glucose levels may be for successive times or for more temporally separated times. For example, predicted glucose levels may be determined that are 5 minutes apart or 15 minutes apart. The predicted glucose level(s) may be determined based on knowledge of the glucose level of the user when the insulin bolus was delivered, the size of the insulin bolus, a correction factor for the user, and JOB. The dosage of the insulin bolus and the correction factor may be used to determine the effect the insulin bolus should have on the glucose level of the user (i.e., the decrease in glucose level due to the bolus is equal to the product of the correction factor and the insulin dosage).

At 1204, one or more glucose readings are obtained from the sensor(s) 106 at predetermined time(s). It may be desirable to obtain multiple readings to account for noise or to check the glucose level of the user over a time window. At 1206, a check is made whether a difference or differences between the glucose level reading(s) and the predicted glucose level(s) is/are over a threshold. For instance, the threshold could be +5% greater than the corresponding predicted glucose level. Other suitable thresholds may be used. The comparison can be between a single glucose level reading and a single predicted glucose level. Alternatively, an average of the glucose level readings may be compared against one of the predicted glucose level values or against an average of the predicted glucose levels. Still further, the glucose level readings may be spread out over time enough such that each is compared to a corresponding predicted glucose level. Where a single glucose level reading or average is compared, if the difference between the value or average and the predicted glucose level or average predicted glucose level is not above the threshold, no further action may be taken as the user is not exhibiting insulin resistance. On the other hand, at 1208, if the difference of the single glucose level reading or the average glucose level reading relative to the predicted glucose level or average predicted glucose level is above the threshold, a notification is generated to prompt the user to action. Specifically, the notification may recommend an activity for the user, such as to exercise. Exercise is known to increase insulin sensitivity. Exercise is believed to affect the beta cells in the pancreas, which enhances insulin sensitivity.

Where multiple glucose level readings are compared separately with respective predicted glucose levels, several options for determining whether the differences are over the threshold at 1206 are possible. First, if any of the differences of the respective glucose levels to the respective predicted glucose level is above the threshold, then a notification may be sent at 1208. If none are over the threshold, no action may be taken. Second, if all of differences of the respective glucose levels to the respective predicted glucose level are above the threshold, a notification may be sent in 1208 and no action may be taken otherwise. Third, if a majority of the differences of the respective glucose levels to the respective predicted glucose level is above the threshold, then a notification may be sent at 1208 and no action taken otherwise.

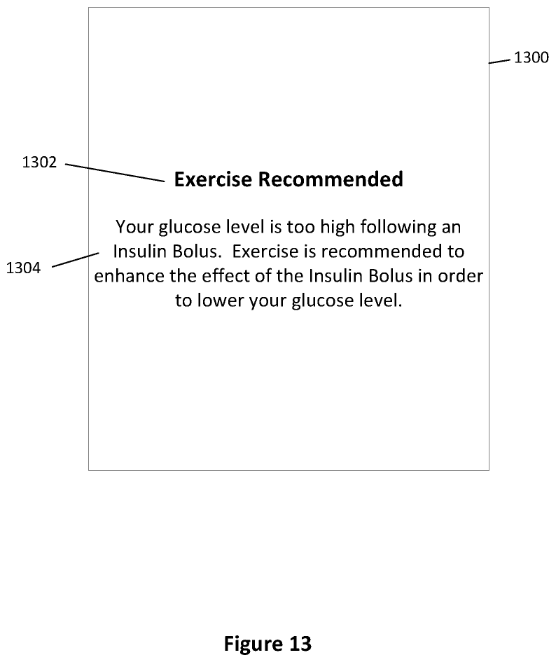

FIG. 13 depicts an illustrative user interface 1300 that may be displayed on display 109 of the medicament delivery device 102, display 127 of the management device 102 or even on smartwatch 130. The user interface 1300 includes a recommendation 1302. In this example, the recommendation 1302 is that exercise is recommended. The user interface 1300 also includes an explanation 1304 for why the recommendation is being made. The user interface may also specify a type and quantity of exercise that is recommended (e.g., run a half mile). The recommendation could recommend other activities other than traditional exercise.

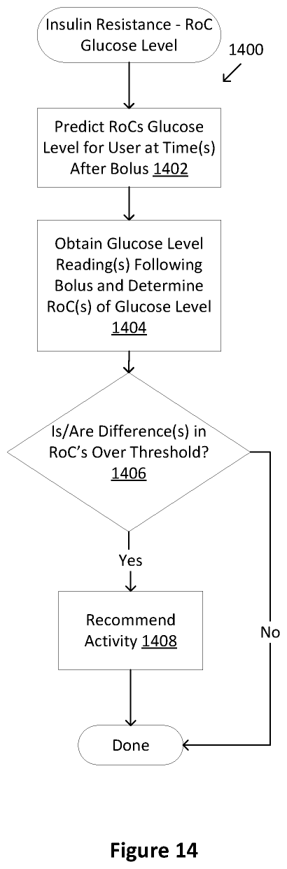

Instead of the glucose level of the user being the key parameter that is checked for possible insulin resistance, the RoC of the glucose level of the user may be checked after delivery of a correction insulin bolus. FIG. 14 depicts a flowchart 1400 of illustrative steps that may be performed in exemplary embodiments to use RoC of the glucose level of the user to identify insulin resistance. At 1402, the RoC(s) of the glucose level of the user at one or more points of time after delivery of a correction insulin bolus is/are predicted. Based the glucose level of the user when the insulin bolus was delivered, the correction factor for the user, the size of the bolus and JOB, a prediction of the RoC(s) of the glucose level(s) can be made. At 1404, a single glucose level reading may be obtained at a single point of time or multiple glucose level readings may be obtained at different points in time after the delivery of the insulin bolus, such as was described above relative to FIG. 12. The glucose level reading(s) may be processed to determine the RoC(s) of the glucose level of the user at the time of each glucose level reading. This may be determined relative to a most recent glucose level reading or the glucose level reading at the time of the insulin bolus.

At 1406, the predicted RoC(s) and the actual RoC at the time(s) may be compared to determine the difference(s). The difference(s) may be compared to a threshold. This may entail calculating a single difference for a single point in time. Alternatively, the comparing at 1406 may entail calculating multiple differences for multiple respective points in time or calculating an average that is compared with an average of the predicted RoCs for the time. Where a single difference (i.e., magnitude and sign) is compared, if the difference exceeds the threshold, at 1408, a notification that recommends activity, like exercise, may be sent. If multiple differences are compared, at 1408, the notification may be sent if one difference exceeds the threshold, if multiple differences exceed the threshold or if all differences exceed the threshold in different embodiments. Otherwise, no action is taken.

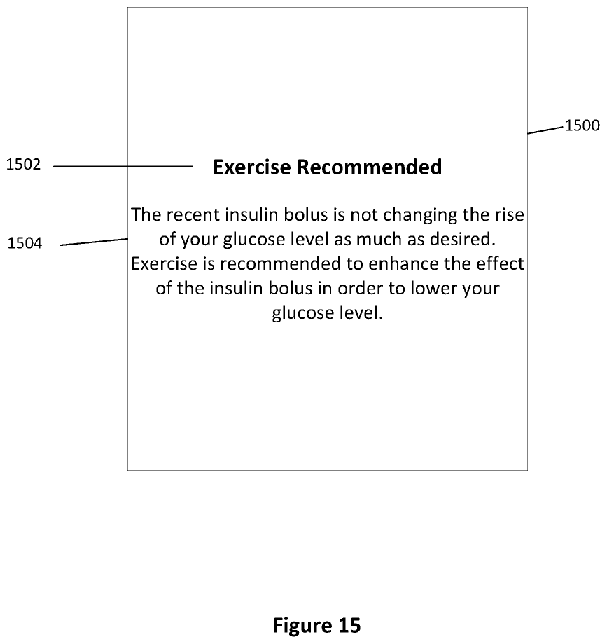

FIG. 15 depicts an illustrative user interface 1500 that may be displayed at 1408. The user interface 1500 may be displayed on display 109 of the medicament delivery device 102, display 127 of the management device 102 or even on smartwatch 130. The user interface 1500 includes a recommendation 1502. In this example, the recommendation 1502 is that exercise is recommended. The user interface 1500 also includes an explanation for why the recommendation is being made. The user interface may also specify a type and quantity of exercise that is recommended.

In some other exemplary embodiments the acceleration or deceleration of the RoC may be used to identify insulin resistance

While exemplary embodiments have been described herein, various changes in form and detail may be made without departing from the intended scope as defined by the appended claims.

1. An electronic device, comprising:
a display for displaying content; and
a processor configured to:
determine projected basal insulin delivery amounts by an automated insulin delivery (AID) device to the user over an interval based on a current rate of change (RoC) of a glucose level of a user, a current amount of insulin on board for the user and a most recent glucose level reading for the user, and
output an indication of the projected basal insulin delivery amounts by the AID device to the user over the interval on the display.

2. The electronic device of claim 1, wherein the indication includes a description of the projected basal insulin delivery amounts by the AID device to the user over the interval.

3. The electronic device of claim 2, wherein the description indicates at least one of the following: that basal insulin delivery to the user will be suspended during at least a portion of the interval, that basal insulin delivery amounts to the user during the interval will be substantially equal to a current basal insulin delivery amount delivered to the user, the basal insulin delivery to the user will be increased during the interval to increased amounts relative to the current basal insulin delivery amount delivered to the user, or the basal insulin delivery amounts delivered to the user will be significantly increased to significantly increased amounts during the interval relative to current basal insulin delivery amount delivered to the user.

4. The electronic device of claim 1, wherein the indication includes graphical content.

5. The electronic device of claim 1, wherein, in determining the projected insulin delivery by the AID device to the user over an interval, the processor is configured to compare the current RoC of glucose level of the user to one or more thresholds, compare the current amount of insulin on board for the user and the most recent glucose level reading for the user to thresholds, and based on the comparing of the current RoC, the comparing of the current amount insulin on board and the comparing of the most recent glucose level reading for the user, determine the projected insulin delivery amounts by the AID device to the user.

6. The electronic device of claim 1, wherein the processor is further configured to determine anticipated glucose levels for the user over the interval in view of the projected basal insulin delivery amounts.

7. The electronic device of claim 6, wherein the processor is additionally configured to determine whether the anticipated glucose levels for the user over the interval will exceed a desired range of glucose levels.

8. The electronic device of claim 7, wherein the processor is additionally configured to notify the user that the anticipated glucose levels for the user over the interval will exceed the desired range of glucose levels.

9. The electronic device of claim 6, wherein the processor is additionally configured to determine whether the anticipated glucose levels of the user over the interval will fall below the desired range of glucose levels.

10. The electronic device of claim 9, wherein the processor is additionally configured to notify the user that the anticipated glucose levels for the user over the interval will fall below the desired range of glucose levels.

11. The electronic device of claim 1, wherein the interval extends from a current time to a time in the future.

12. The electronic device of claim 12, wherein the time in the future is a time in a range of 15 minutes after the current time to two hours after the current time.

13. An electronic device, comprising:
a display for displaying content; and
a processor configured to:
determine projected insulin delivery amounts by an automatic insulin delivery (AID) device to a user over a future interval,
determine if anticipated glucose levels of the user for the future interval stay within a desired range, and
if the anticipated glucose levels of the user for the interval are determined to not stay within the desired range, output a recommendation on the display for action by the user to adjust their glucose level.

14. The electronic device of claim 13, wherein the recommendation is for the user to receive a correction insulin bolus and/or to exercise.

15. The electronic device of claim 13, wherein the recommendation is for the user to consume carbohydrates.

16. The electronic device of claim 13, wherein the electronic device is at least one of a management device for the AID device, the AID device, a smartphone, a wearable device or a handheld device.

17. A method performed by a processor of an electronic device that includes a display, comprising:
determining projected basal insulin delivery amounts by an automatic insulin delivery (AID) device to a user over a future time window;
determining anticipated glucose levels of the user over the future time window in view of the determined projected basal insulin delivery amounts; and
displaying a user interface on the display, wherein the user interface includes graphical content regarding the anticipated glucose levels and a visual cue of the projected basal insulin delivery amounts to inform the user of the projected basal insulin delivery amounts.

18. The method of claim 17, wherein the determining of the projected basal insulin delivery amounts is based on a current glucose level of the user, a current rate of change of glucose level of the user and total daily insulin for the user.

19. The method of claim 17, further comprising determining that at least some of the anticipated glucose levels over the future time window will exceed a threshold and in response, outputting a recommendation on the display of action that the user may take to reduce the anticipated glucose levels over the future time window.

20. The method of claim 17, further comprising determining that at least some of the anticipated glucose levels over the future time window will fall below a threshold and in response, outputting a recommendation on the display of action that the user may take to increase the anticipated glucose levels over the future time window.
